(19) United States
(12) Patent Application Publication
(10) Pub. No.: US 2002/0168692 A1
(43) Pub. Date: Nov. 14, 2002

(54) Biosensor detector array

(76) Inventor: Anthony E.G. Cass, London (GB)

Correspondence Address: NIXON & VANDERHYE P.C., 8th Floor, 1100 North Glebe Road, Arlington, VA, 22201-4714, US

(21) Appl. No.: 10/055,367

(22) Filed: Jan. 25, 2002

Related U.S. Application Data
(63) Continuation-in-part of application No. PCT/GB00/03768, filed on Oct. 2, 2000.

(30) Foreign Application Priority Data
Sep. 30, 1999 (GB) GB9923146.6

(51) Int. Cl. G01N 33/53; G01N 33/542; C12M 1/34

(52) U.S. Cl. 435/7.9; 435/287.2

(57) ABSTRACT

A method is provided for analyzing a sample. The method comprises the steps of: i) contacting the sample with a detector array comprising a plurality of discrete biological sensing elements immobilized onto or within a solid support; wherein each discrete biological sensing element comprises a detectable label whose characteristics change detectably when the element binds to a ligand within the sample; ii) measuring the characteristics of the detectable label for each element of the array to produce a pattern; and iii) performing data analysis of the pattern; wherein the biological sensing elements are capable of binding more than one different ligand.

This application is a CIP of PCT/GB00/03768 filed on Oct. 2, 2000, the content of which is incorporated herein by reference.

### FIELD OF THE INVENTION

The present invention relates to detector arrays comprising biological sensing elements with broad spectrum ligand specificity. These arrays are useful in methods of analyzing complex mixtures of ligands such as clinical samples or cell extracts, as well as gaseous or volatile substances of both biological and non-biological origin.

### BACKGROUND TO THE INVENTION

The majority of biological detection systems used to date rely on highly specific reactions between detection elements, such as antibodies, and their target ligands. An example of such an approach is the use of antibodies to measure the levels of chorionic gonadotrophin (hCG) in the urine in order to detect pregnancy. However, such systems have a relatively narrow information content since they are designed to recognize only specific reactions between a specific ligand and a specific detection element to generate a positive/negative result. Indeed, great attempts have been made to increase the specificity of such systems and reduce non-specific interactions to reduce the occurrence of false positive and false negative results.

Thus, these highly specific detection systems are capable of generating only a limited amount of information such as the level of hCG in a urine sample. To increase the information content of these detection systems requires a large number of properly characterized and highly specific detection elements, since there is a direct relationship between the amount of information and the number of elements required. Consequently, this approach lacks flexibility and can be expensive if a large number of different detection elements are required.

An alternative strategy is mentioned in WO 97/49989. In this arrangement a detector array is created using lectins as sensing elements. A group of lectins is created within an array, each lectin discrete from and structurally distinct from one another and each having a different affinity and specificity for a ligand. Because lectins recognize only carbohydrates, ligands under investigation using this array must either inherently contain an appropriate carbohydrate or must be bound to an appropriate carbohydrate. Moreover, WO 97/49989 describes a non-label technique which relies on determining an increase in the mass of the sensing elements as a measure of ligand binding. Detection of ligand binding is made using surface plasmon resonance. There are many limitations to this method, including the fact that the requirement for the ligand to contain the appropriate carbohydrate for binding the lectin sensor prevents this array from performing analyses of low molecular weight compounds or compounds which differ from one another in structure but are virtually identical in molecular weight, particularly where the compounds share a common carbohydrate moiety.

Furthermore, non-label detection techniques require sophisticated apparatus that are difficult to use. WO 97/49989 acknowledges that surface mass-based imaging technology was very difficult to use with the biological sensing elements and required the development of a highly specialized protocol for immobilizing the elements onto the solid substrate.

Consequently, there is a need for an improved, broad specificity detection system.

### SUMMARY OF THE INVENTION

A detector array is provided which comprises a variety of broad specificity or promiscuous sensing elements and variants thereof, all of which sensing elements and variants have attached thereto a detectable label. Application of a ligand(s) to the detector array and binding of the ligand(s) to the sensing elements and/or variants thereof causes a detectable change in the properties of the label depending on the nature of the interaction between the ligand(s) and the sensing element/variant, measurement of which allows collection of data for identification or “fingerprinting” of the ligand(s). The invention also provides the use of a detector array in the identification of a sample ligand, as well as methods for the formation of the detector array.

### BRIEF DESCRIPTION OF THE FIGURES

FIG. 1 is a diagram illustrating the formation of variants of the biological sensing elements and their incorporation into the detector array of the present invention.

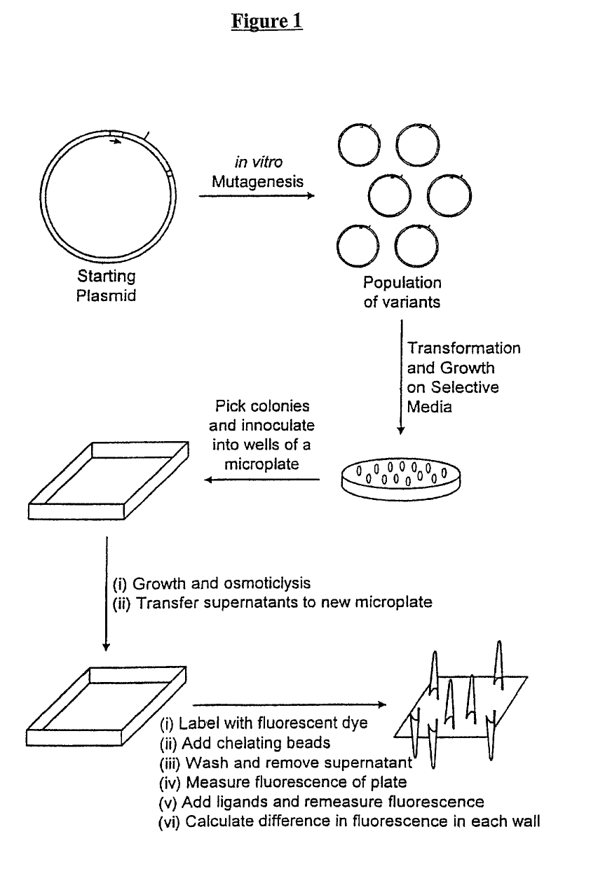

FIG. 2 is a graph illustrating the ability of the detector array of the present invention to distinguish between low molecular weight compounds of very similar structure. The graph shows the change in the fluorescence intensity caused by binding of the ligand (thymol/menthol) to a sensing element (immobilized bovine olfactory protein) site specifically labeled with acrylodans.

FIG. 3 is a bar graph showing the different fluorescence intensity at each of four mutants of a biological sensing element (bovine olfactory binding protein) caused by binding of three ligands (menthol, isomenthol and thymol) to those mutants. This figure shows that three low molecular weight compounds having close structural similarity (two of which are positional isomers of each other) can be distinguished using the detector array of the present invention.

FIG. 4 illustrates the discriminating ability of the six odorant binding protein variants for low molecular weight compounds of very similar structure. The graph shows that the change in the fractional saturation of the binding site of the protein variants by thymol and menthol varies with changes in ligand concentration. This indicates that the affinity-binding profile of the ligands to the sensing elements shown is both concentration and ligand dependant.

FIG. 5A is a bar graph showing the different normalized fluorescence intensity at each of five mutants of an olfactory binding protein caused by binding of five ligands (all NSAIDS) to those mutants. This figure illustrates the different binding profiles that compounds sharing a common biological or pharmacological activity demonstrate.

FIG. 5B shows a principal component analysis of the binding patterns of 17 sample compounds passed over the detector array. The 17 compounds cluster into three groups based upon analysis of their data array patterns.

### DETAILED DESCRIPTION OF THE INVENTION

Accordingly in its first aspect the present invention provides a detector array comprising one or more groups of broad specificity biological sensing elements and variants thereof discretely immobilized onto or within a solid support, wherein the sensing elements and variants thereof have attached thereto a detectable label.

In an embodiment of this aspect of the invention there is provided a detector array comprising a plurality of discrete biological sensing elements immobilized onto or within a solid support wherein:

(a) each sensing element has a ligand binding site capable of binding a broad range of structurally diverse ligands;

(b) the sensing elements are provided in groups, each group comprising a biological sensing element and at least one variant thereof, said variant differing from the element from which it was derived in its ligand binding specificity and/or affinity; and

(c) each sensing element and variant thereof having a detectable label attached thereto, the physical characteristics of said label being susceptible to change upon ligand binding.

In its second aspect, the present invention provides a method for providing a detector array system comprising:

(a) contacting a detector array as hereinabove defined with a panel of known test ligands;

(b) measuring the characteristics of the detectable label for each sensing element and variant thereof upon ligand binding to produce a data array pattern; and

(c) using the data array pattern to generate a reference database of said patterns.

In furtherance of the above second aspect, the method defined above may be supplemented by:

(d) contacting the array with a sample containing a sample ligand;

(e) producing a data array for the sample ligand; and

(f) comparing the data array for the sample ligand with the reference database of data array patterns obtained from said test ligands.

The invention is described in more detail in sections (i) to (xii) below, and by reference to the accompanying examples and drawings. Sections (i) to (xii) below are:

i) Biological Sensing Elements

ii) Ligand Binding Site

iii) Ligands

iv) Interaction between sensing element and ligand

V) Preparation of Sensing Elements

vi) Arrangement within the Detector Array

vii) Variants

viii) Detectable Labels

x) Detector Array Assembly

x) Assay System

xi) Measurements

xii) Computer Systems

i) Biological Sensing Elements

The detector array of the present invention comprises a plurality of discrete biological sensing elements. As may be seen from the above, one aspect of the invention is to prepare a database of the patterns produced or binding profiles resulting from contacting the detector array with a panel of known ligands, and then to compare the binding profile of a sample ligand run on the same detector array with that database.

The term “biological sensing element” as used herein is intended to mean a receptor for another molecule, typically a ligand, the biological sensing element having the ability to bind a ligand, usually in a reversible manner. The term “biological” refers to the nature of the sensing element and not the nature of the ligand which binds to it, which may be either biological, such as pathogen-derived proteins, or non-biological, such as petrochemicals or an organic molecule, such as a putative drug.

The term “discrete” in relation to the biological sensing elements and/or variants thereof means that each element/variant is placed on or in the detector array such that the spacing between each element/variant allows the signal from each individual element/variant to be resolved by the detection equipment.

The number of discrete biological sensing elements in the detector array will be that sufficient to enable formation of a reference database such that that database contains sufficient information to allow accurate identification of a test compound. Typically, the detector array will consist of at least two sensing elements, preferably from 2 to 250 sensing elements and most preferably from 25 to 250 sensing elements.

Preferably sensing elements are proteins/polypeptides or fragments thereof which are of small size. Typically, the sensing element is less than 200 kDa in weight, preferably less than 100 kDa and most preferably 50 kDa or less in weight.

These sizes may be estimated using techniques known in the art, for example using polyacrylamide gel electrophoresis, or they may be calculated molecular weights derived from a knowledge of the gene or protein sequence or structure.

It will be understood that candidate sensor polypeptides for use in the present invention are not restricted to comprising full length naturally occurring polypeptides. Fragments, truncations, domains (whether singly or in combination) or concatenations of such molecules may be utilized, or, in the alternative, polypeptide molecules having a lipocalin fold may be used, with the proviso that in each case, the sensing element includes a ligand binding site. At the minimum, the sensing element can consist of the ligand binding site alone, although it is preferred that substantially more of the polypeptide from which the ligand binding site was derived also forms part of the sensing element. Thus, it is preferred that those sequences of the polypeptide which are required to maintain the conformation of the binding site also form part of the sensing element.

Sensing elements for use in the detector array of the present invention typically have broad specificity. They may otherwise be described as being promiscuous. By broad specificity or promiscuity is meant that each sensing element is capable of binding a wide variety of different ligands. The binding affinity of the sensing element for different ligands may vary. Preferably the sensing elements are capable of binding a broad range of structurally diverse ligands. Broad specificity or promiscuity may also be understood to relate to the structural determinants of the ligand(s) to which the sensing elements bind. Although not every sensing element in the detector array need have broad specificity or be promiscuous, it is preferred that substantially all of the elements possess such broad specificity/promiscuity.

The choice of sensing element for use in the detector array of the present invention will be influenced to a certain extent by the ligand to be tested. It is within the skill of the person in the art to construct an array using a biological sensing element which is known to bind the class of ligand under investigation. Of course, sensing elements having broad specificity or being promiscuous will be useful in the determination of a variety of different ligands.

Exemplary polypeptides which are naturally promiscuous or which inherently have broad specificity and which are hence useful as sensing elements include mammalian or insect olfactory binding proteins, membrane-bound proteins, chaperone proteins (e.g. Gα15 and Gα16), PXR receptor, taste receptors, DNA binding proteins, serum albumin (human and bovine), cytochrome P450's, P-glycoprotein, major urinary protein and others. Polypeptides into which promiscuity can be introduced for example by chemical and/or mutagenic modification and which can hence also be useful as sensing elements include bacterial periplasmic binding proteins, such as maltose binding proteins, phosphate binding proteins, glucose-galactose binding proteins, arabinose binding proteins and glutamine binding proteins. Particularly preferred are the mammalian olfactory binding proteins and most preferred of this group is the human olfactory binding protein (hOBP), bovine olfactory binding protein (bOBP) or the porcine olfactory binding protein (pOBP). Especially preferred is bOBP. Olfactory binding proteins are well known in the art and may be obtained either from publicly available sources or using known procedures. Specifically, OBPs may be obtained by reference to the GENBANK or EMBL databases. For example, GENBANK accession number [NM 014581] is an hOBP. The protein sequences of bOBP and pOBP are known and these sequences can be obtained by reference to EMBL accession numbers P07435 and P81245 respectively. Other molecules may be employed as sensors in the present invention, such as avidin. Combinations of polypeptide and non-polypeptide molecules may be employed as sensors in the present invention, such as maltose binding protein complexed with cyclodextrin or other host molecules. This combination is particularly advantageous in the study of non-steroidal anti-inflammatory compounds as ligands.

ii) Ligand Binding Site

Sensing elements are typically proteins, polypeptides or fragments thereof comprising a ligand binding site. Although the ligand binding site need not be specifically characterized in terms of its amino acids, it is preferred that at least the location of the ligand binding site within the polypeptide is known, and it is more preferred that at least some information on amino acids with respect to the ligand binding site is known. Determination of the ligand binding site within a polypeptide may be made using techniques common in the art, for example X-ray crystallography. Determination of the specific amino acids in a ligand binding site may likewise be made using techniques common in the art.

Each sensing element for use in the detector array of the present invention has a ligand binding site capable of binding a broad range of structurally diverse ligands. As defined above, such a sensing element is termed promiscuous or as having broad specificity. A broad specificity/promiscuous sensing element preferably binds a number of ligands, which ligands may apparently lack a common structural determinant, such as a conserved functional group(s) or particular size or shape of molecule, which shared features by contrast, usually define classes or types of molecules (ligands) binding to highly specific sensing elements used in other arrays. An example of a protein which may itself form a sensing element or from which a sensing element may be derived is porcine olfactory binding protein (pOBP). This protein has broad specificity and as such is a preferred sensing element. Ligands known to bind to pOBP include benzophenone, benzylbenzoate, dihydromyrcenol, IBMP (isobutylmethylpyrazine), selenazol, thymol and undecanal. Clearly a comparison of the chemical structures of these diverse compounds illustrates the meaning of the term broad specificity. Furthermore, an exemplary characteristic of the term broad specificity as explained above is the lack of common chemical and/or structural features between the various ligands which bind to the sensing element. This characteristic is clearly demonstrated in the example of pOBP, since other than the most basic structural feature of being low molecular weight organic molecules, there is no obvious common structural determinant between the various ligands binding pOBP.

Biological sensing elements for use in the detector array of the present invention are typically chosen for their ability to bind ligands of low molecular weight.

iii) Ligands

The term “ligand” used herein refers to any compound which may be able to interact, to any extent, with a biological sensing element of an array of the invention. It will be understood that the term does not imply that a compound is the “natural” ligand for the sensing element. The use of broad specificity sensing elements and variants thereof will allow a broad range of different chemical entities to bind.

The terms “test ligand” or “known ligand” refer to ligands which have a known structure and/or biological property of interest. In performing the present invention, a starting point is the provision of a set of test ligands with which to train or calibrate an array.

A “sample ligand” refers to a ligand whose identity is, or is suspected to be, previously encountered by a trained array of the invention. Thus an array of the invention may be used to identify a sample ligand by comparing its binding pattern with the binding patterns of test ligands whose identity has previously been established.

A “candidate ligand” is a ligand which is to be investigated for a desired biological property by comparison of its array binding pattern with the array binding pattern of test ligands, at least some of which are known to have the biological property in question. In this aspect of the invention, it is not necessary for the array to have previously encountered the candidate ligand; rather the property of the ligand is inferred by the neural network, multivariate statistical or pattern recognition analysis software associated with the array through its learned knowledge of binding profiles common to members of the test ligand set which share the biological property of interest. In the accompanying examples, we demonstrate that five NSAID (non-steroidal anti-inflammatory drugs) can be discriminated from two other classes of compounds by principle component analysis (PCA).

Typically the ligands which may be used in accordance with the invention will be of from 32 to 600 Da, preferably from 50 to 500 Da and most preferably of from 100 to 400 Da. This reflects the typical molecular weight range of many small molecule drugs.

Candidate ligands may be obtained from commercially available libraries of compounds, or generated de novo using, for example combinatorial chemistry (see, for example A Practical Guide to Combinatorial Chemistry, A. W. Czarnik & S. H. DeWitt, American Chem. Soc. 1997 and Combinatorial Chemistry, N. K. Terrett, OUP, 1998). Commercially available libraries of compounds are available from, for example, Sigma-Aldrich-Fluka, Chemical Diversity Incorporated and Tripos.

Examples of low molecular weight ligands which can be recognized using the detector array of the present invention are classes of drug, such as angiotensin converting enzyme inhibitors, beta-adrenergic inhibitors and non-steroidal anti-inflammatory agents; general classes and functional derivatives of organic molecules, such as aromatic and aliphatic alcohols, aldehydes and ketones; natural products such as terpenes, and simple sugars.

iv) Interaction Between Sensing Element and Ligand

The character of the interaction between ligand and sensing element can be a further or an alternative indicator of the broad specificity of the sensing element in that binding to broad specificity sensing elements of the present invention is not generally of the “lock and key” type associated with, for example, an enzyme-substrate interaction, which often demands a very precise spatial fit of the ligand (e.g. substrate) into the element (e.g. the enzyme molecule). The mechanism of binding of broad specificity sensing elements of the present invention to the ligands is preferably less rigid, and resembles a dissolution of the ligand into the binding site rather than a fixed three-dimensional co-ordination of particular chemical groups of the ligand. Thus, the interaction of ligand with a broad specificity sensing element of the present invention is less likely to be absolutely dependent on a particular feature of the sensing element, such as a particular amino acid residue, but is more likely to be affected to a smaller degree by mutations, for example by a change in the binding affinity or a change in the profile of ligands to which it will bind, rather than an absolute abolition (or restoration) of binding by a single mutation as can be found in other arrays, for example those used to map receptor-ligand interactions.

A broad specificity sensing element of the present invention is thus less likely to bind its various ligands through hydrogen bonding, salt bridges and the like, but is more likely to retain the ligand through energetic considerations such as entropy change due to displacement of protein bound water or via a large number of weaker bonding forces. These can include van-der Waals and/or hydrophobic-hydrophilic dipolar interaction as contrasted with, for example, the directional hydrogen bonding exhibited by other ligand-sensor interactions.

A broad specificity sensing element of the present invention is less likely to have a binding site which is precisely defined with respect to amino acid residues which may co-ordinate ligand binding, but is more likely to have a binding site which is less specifically defined, or may be defined geometrically (e.g. defined as an area or surface or pocket on the polypeptide, rather than defined chemically by reference to particular amino acid residues). Thus different ligands, whilst binding in the same general region of the broad specificity sensing element, interact in that binding with different amino acid residues.

Binding characteristics of the sensing elements may be investigated using one or more known ligand(s). This may comprise a panel of ligands or may comprise one or a number of candidate molecules binding a particular polypeptide sensor. In the case of bovine olfactory binding protein (bOBP), an example of a suitable test ligand is thymol. Other appropriate test ligands for bOPB include camphor, decane or any other analyte of interest. In the case of maltose binding protein as sensing element, suitable ligands include maltose and cyclodextrin. In the case of a maltose-cyclodextrin combination used as sensor, suitable ligands include non-steroidal anti-inflammatory compounds.

v) Preparation of Sensing Elements

The sensing elements for use in the detector array of the present invention may be prepared by any appropriate means. Preferably the sensing elements are proteins/polypeptides or fragments thereof which can be expressed (typically these are readily over-expressed) in a suitable host organism, such as a micro-organism, typically E. coli. This is a standard procedure well known to those skilled in the art, and is discussed in more detail below. Briefly, nucleic acid encoding the polypeptide is cloned into an expression vector and this expression vector is transformed into a host strain of E. coli for protein expression. Expression is induced, and preferably proteins/polypeptides or fragments thereof suitable for use as sensing elements of the present invention are highly expressed, and preferably readily extracted or purified as discussed in the Example section. Less preferred are polypeptides which form insoluble inclusion bodies on expression and which require alternative extraction techniques and in vitro refolding.

vi) Arrangement within the Detector Array

The biological sensing elements for use in the detector array of the present invention are arranged in groups, each group comprising a sensing element and at least one variant thereof.

Groups are generally defined in terms of the relationship between the sensing element and the variants thereof. Although not every member of the group need be a variant of one of the other members of the group, it is preferred that substantially all members of the group are variants of the sensing element forming part of that group. Whilst it is not essential that individual members of a group are in physical proximity to one another within the array, it is generally preferred that individual members of a group are so arranged in the array that there is physical proximity between the members.

A group typically comprises at least two members, preferably at least three or four members. Most preferably a group comprises one sensing element and from 1 to 100 variants thereof, preferably from 2 to 40 variants thereof and most preferably from 5 to 25 variants thereof.

In the detector array of the present invention there is generally at least 1 group of sensing elements and variants thereof. Preferably there are at least 2 groups and most preferably there are from 2 to 50 groups.

vii) Variants

The detector arrays of the present invention include biological sensing elements and variants thereof. A variant of a biological sensing element is derived from that biological sensing element and differs from the biological sensing element in its binding specificity and/or affinity.

When the biological sensing element is a polypeptide, variants thereof may contain up to 10, preferably from 1 to 5, most preferably from 2 to 4 points of difference within the amino acid sequence, preferably of the ligand binding site, from the biological sensing element from which they are derived.

The precise number of points of difference between a variant and the biological sensing element from which it was derived will differ depending on the location of the detectable label within the variant and on the means by which the variant is to be immobilized onto or within the array, as well as on modifications to the sensing element introduced to effect the specificity/affinity of the resulting variant. Generally modifications to effect the specificity/affinity of the variant will occur in the ligand binding site, although they may also occur outside the ligand binding site but in a position which has an effect on the specificity/affinity of the resulting variant.

Variants in each group in the array are typically obtained by modification of a primary element in the biological sensing element to alter its binding specificity and/or affinity. It is preferred that the sensing element is able to tolerate alterations, that is it is preferred if the sensing elements can be altered and/or mutated as herein described without totally destroying the activity of the protein, for example by causing misfolding, insolubility or loss of function of one of more preferred characteristics as discussed herein. Within a group formed from a sensing element and variants thereof, there can be variants which do not retain any of the binding characteristics of the sensing element from which they were derived, but which are still capable of binding a ligand.

In a polypeptide sensing element, modification may be of one or more amino acids within the ligand binding site, or may be of one or more amino acids outside the ligand binding site. In either case, the modification will have an effect on the binding specificity/affinity of the variant thereby created. Modifications to the sensing element not falling within the binding site will typically cause a change in the folding of the polypeptide, thereby altering the binding specificity/affinity of that polypeptide for a ligand. Modification may be both within and outside the ligand binding site. Modification may be by chemical means, using reagents and conditions known in the art. The chemical modification will typically result in a change in the structure of the biological sensing element, whereby the precise nature of the resulting change in specificity and/or affinity does not need to be specifically characterized before use of the variant in the detector array of the present invention.

Variants of the biological sensing element may also be obtained by mutagenesis. Mutagenesis techniques include site-directed mutagenesis of the ligand binding site or of the areas bordering the ligand binding site or any other part of the biological sensing element that results in a structural change affecting the binding specificity/affinity of the resulting variant. Alternative techniques include domain swapping, whereby using standard cloning technology, sections of an element are replaced with sections from a related or unrelated polypeptide. Mutagenesis includes insertions, deletions and substitutions. Amino acids may be non-naturally occurring amino acids to increase the structural diversity.

A particularly preferred method for mutagenizing biological sensing elements is to amplify the gene for the biological sensing elements by the polymerase chain reaction under conditions where there are random mistakes made in the nucleotides being incorporated. The conditions under which such “error prone PCR” occurs are well known to those skilled in the art. The mixture of randomly mutated genes is then inserted into an appropriate vector, transformed into a host and followed by screening the resulting bacteria or viruses using standard techniques (such as expression screening or phage display). The polynucleotide encoding the sensing element conveniently also encodes a reporter fusion protein in frame with the sensing element construct to allow easy identification of the mutagenized proteins over other bacterial/viral proteins. As discussed above, the polynucleotide encoding the sensing element comprises a sequence encoding an affinity tag such that the affinity tag is produced in frame at the C-terminus of the sensing element. Mutagenized proteins may be purified directly from the bacteria/viruses or the polynucleotide constructs obtained and cloned into other suitable vectors/hosts for expression and purification of the biological sensing elements or variants thereof.

In the alternative, variants of the sensing elements can be prepared by a combination of chemical modification and mutagenesis techniques.

The specific modifications introduced into the sensing element to form a variant need not be characterized. However, one modification which is preferred in the creation of a variant is the introduction, relocation or removal of a cysteine residue(s) within the target area. The target area may be defined as the ligand binding site of the biological sensing element and/or residues affected by binding of a ligand. These latter residues may include that part of the polypeptide to which a detectable label is to be attached and to which modification by inclusion of a cysteine residue will result in a change in the specificity/affinity of variant from the biological sensing element from which it is derived.

Thus, for example, when the biological sensing element chosen is a polypeptide containing a ligand binding site of, for example, X amino acid residues, none of which is naturally a cysteine residue, X variants may be created by substituting each of the amino acid residues of the binding site in turn for a cysteine residue.

Typically, when the biological sensing element does not contain any cysteine residues within the target area, only one cysteine residue will be introduced into the target area or, where a cysteine residue already exists within the target area of the biological sensing element, if that cysteine residue forms part of a disulphide bond, it may be left where it is. If the cysteine residue in question is not part of a disulphide bond it may be removed or relocated within the target area of the biological sensing element, but no further cysteine residues will be introduced. Alternatively, if the presence of a single naturally occurring cysteine residue is desirable in the target area of the sensing element, that cysteine residue may be left where it is. In the case that the target area of the sensing element naturally contains more than one cysteine residue, the man skilled in the art will readily be able to determine whether the presence of multiple cysteine residues within the target area is desirable. Thus, for example, cysteine residues involved in the formation of a disulphide bond will be retained as having an effect on the three-dimensional structure of the protein. It is generally preferred that all bar one cysteine residue are removed from the protein and replaced with other amino acids.

In a preferred embodiment, the ligand binding site of a variant of a biological sensing element for use in the detector array of the present invention will include one cysteine residue, which residue may either be naturally occurring or which may have been introduced.

viii) Detectable Labels

The biological sensing elements and variants thereof for use in the detector arrays of the present invention have a detectable label attached thereto. The physical characteristics of that label are susceptible to change upon ligand binding. By change is meant that the characteristic is altered, modulated or otherwise affected by the binding of a ligand. The detectable label is preferably a fluorescent label. The change in the physical characteristics of the label is typically detected by optical or electrical means, for example when the label is a fluorophore by a change in the emission intensity, excitation or emission wavelength, excited state lifetime, a change in absorption characteristics and/or polarization, or any other measurable characteristic. Preferably the detection is via optical means.

The label may be attached at any appropriate position within the sensing element, such that the physical characteristics of the label will be changed upon binding of the ligand. Thus, the label may be attached within the ligand binding site, so long as it does not interfere with ligand binding, or the label may be attached outside the ligand binding site, but in either case is so positioned that ligand binding causes a change in the physical characteristics of the label. Within a group formed from a biological sensing element and variant(s) thereof, the detectable label may be attached at the same or different sites within the various members of the group.

Furthermore, the positioning of a label at different sites for example in the ligand binding site of a sensing element and/or variant thereof may have an effect on the specificity/affinity of that sensing element and/or variant for ligand binding. By positioning the label at differing sites throughout the sensing element and/or variant thereof, further variants are effectively created.

A detectable label may be attached to the sensing element or variant thereof using techniques familiar to the man skilled in the art. In one example, the detectable label may be attached to the sensing element or variant thereof via a cysteine residue. The cysteine residue may be naturally occurring or may have been introduced, as discussed above. Suitable location(s) in polypeptide sensing elements for the introduction of cysteine residues for fluorescent labeling may be chosen by a person skilled in the art, preferably placing them so that they do not interfere with the binding of the ligand. Such cysteine residues are preferably placed on or near residues which move and/or change conformation on ligand binding. In addition or alternatively, such residues are preferably placed at a location which will not interfere with expression/purification/immobilization of the sensing element. A further preference in the siting of a cysteine residue is at a position whose exposure to solvent is altered (i.e. increased or decreased) as a consequence of ligand binding.

In a highly preferred embodiment of the present invention, each of these preferences is satisfied in the placement of a cysteine residue for reaction with a fluorophore.

If sufficient information is not available to make meaningful choices about the placement of a cysteine residue for fluorophore labeling a priori, a simple trial-and-error approach may be used, making a number of variants and picking the variant with the cysteine location resulting in the sensing element with the most suitable characteristics as described herein.

The biological sensing elements and variants thereof are linked to a detectable label such that when the sensing element/variant binds a ligand, there is a detectable change in a characteristic of the label.

When, for example, the label is a fluorophore, a change in a fluorescent property, for example intensity, excited state lifetime, excitation or emission wavelength or polarization may occur upon ligand binding.

Preferably the label is a fluorescent group with excitation and/or emission wavelength in the optical spectrum (350 to 750 nm). More preferably the label shows an increase in emission intensity and/or shift in emission wavelength.

Examples of fluorescent probes which vary among themselves in excitation and emission maxima are listed in Table 1 of WO 97/28261. These (each followed by [excitation max./emission max.] wavelengths expressed in nanometers) include wild-type Green Fluorescent Protein [395(475)/508] and the cloned mutant of Green Fluorescent Protein variants P4 [383/447], P4-3 [381/445], W7 [433(453)/475(501)], W2 [432(453)/480], S65T [489/511], P4-1 [504(396)/480] S65A [471/504], S65C [479/507], S65L [484/510], Y66F [360/442], Y66W [458/480], Ioc [513/527], W1B [432(453)/476(503)], Emerald [487/508] and Sapphire [395/511]. This list is not exhaustive of fluorescent proteins known in the art: additional examples are found in the Genbank and SwissProt public databases.

Alternatively fluorophores such as fluorescent dyes may be used. Examples of fluorescent dyes include the following non-limiting list of chemical fluorophores provided, together with their emission wavelengths, in Table 1.

TABLE 1

| Fluorophore | Excitation (nm) | Emission (nm) | Color |
|---|---|---|---|
| PKH2 | 490 | 504 | Green |
| PKH67 | 490 | 502 | Green |
| Fluorescein | 495 | 525 | Green |
| (FITC) | | | |
| Hoechst 33258 | 360 | 470 | Blue |
| R-Phycoerythrin | 488 | 578 | Orange-red |
| (PE) | | | |
| Rhodamine (TRITC) | 552 | 570 | Red |
| Quantum Red ™ | 488 | 670 | Red |
| PKH26 | 551 | 567 | Red |
| Texas Red | 596 | 620 | Red |
| Cy3 | 552 | 570 | Red |

A particularly preferred group of labels for use in the array of the present invention include the so-called fluorescent probes. These may be defined as being environmentally sensitive, such that changes in, for example, the pH or polarity of the environment, potentially caused by ligand binding to the sensing element/variant, will have an effect on the properties of the probe. Such fluorescent probes are also less sensitive to the non-specific binding background and enable quantitative measurements to be made. Thus, they are capable of reporting the mechanism of binding of a ligand rather than simply being switched on or off by binding of the ligand.

When the label is a fluorescent probe, it is generally preferred that the probe has at least some and preferably all of the following properties:

1) Low molecular weight, for example from 150 to 500 Da;

2) It can be conjugated in a site-specific manner to thiol groups that have been introduced into or are naturally occurring in the target area of the sensing element;

3) It exhibits an enhanced fluorescence over that observed in free solution when conjugated with thiol groups that are buried or unsolvated, for example cysteine groups present or introduced in the sensing elements/variants of the present invention;

4) It exhibits a detectable shift in fluorescence emission spectra upon binding of ligand to a sensing element/variant.

Examples of fluorescent probes suitable for use as labels in the array of the present invention include the following non-limiting list, provided in Table 2.

TABLE 2

| Fluorescent Probe | Excitation (nm) | Emission (nm) |
|---|---|---|
| Iodoacetylnitrobenzoxadiazole | 482 | 520 |
| Acrylodans | 360 | 430-550 |
| Iodoacetamidobenzoxadiazole | 482 | 525 |

Further fluorescent probes suitable for use include but are not limited to coumarins, anilinonaphthalenesulphonate iodoacetamide (IAANS) and maleimide (MIANS) and 5-{([(2-iodoacetyl)amino]ethyl)amino}naphthalene-1-sulfonic acid (IAEDANS).

Although the acrylodan dyes generally react with thiol groups more slowly than do the IAANS and MIANS compounds, they form very strong thioether bonds that are typically stable under most reactive conditions. The fluorescence emission peak and intensity of these adducts are particularly sensitive to conformational changes or ligand binding.

Most acrylodan dyes suitable for use as a detectable label in the array of the present invention have their longest-wavelength absorption peaks at less than ˜400 nm. Typically these dyes exhibit blue fluorescence and have weak absorption, with extinction coefficients often below 20,000 cm⁻¹M⁻¹. Photostability of UV light-excitable dyes is typically is less than that of visible light-excitable dyes. The strong dependence of the emission spectra and quantum yields of several of the dyes makes them useful for studying ligand binding to receptors. The spectra of certain dyes tend to be particularly sensitive to ligand and metal binding, protein association and chaotropic reagents. When protein conjugates of these dyes are denatured or undergo a change in conformation, a decrease in fluorescence intensity and a shift in emission to longer wavelengths are often observed.

A further fluorescent probe suitable for use as a label in the array of the present invention is IANBD. This compound contains donor-acceptor electron pairs and can form a twisted intramolecular charge transfer (TICT) excited state, both of which ensure that excited state relaxation is sensitive to rotational freedom and/or salvation. The intensity of fluorescence emission of IANBD is highly sensitive to changes in the salvation level of the fluorophore. Upon ligand binding to conjugated amino acid residues, the probes localized electronic environment is disturbed resulting in a quenching or enhancement of fluorescence being seen. The nature of this change is reflective of both the structure of the bound ligand, the nature of the interaction (hydrophobic, van-der Waal's, dipolar etc) and the nature of the assay medium (pH, ionic strength).

Further suitable fluorescent probes for use in the arrays of the present invention include IAANS and MIANS. To develop appreciable fluorescence, both the reactive IAANS and MIANS must be reacted with thiols that are located in hydrophobic sites. Often, however, buried unsolvated thiol residues are exceptionally reactive, allowing these sites to be selectively modified by these reagents. The environmentally sensitive fluorescence properties of the protein conjugates of MIANS and IAANS are similar to those of IANBD. The fluorescence intensity, and to a lesser extent the emission wavelengths, of the conjugates tends to be very sensitive to substrate binding, folding and unfolding of the protein and changes in local polarity. Like most other maleimides, MIANS is essentially non-fluorescent until it has reacted with a thiol.

IAEDANS is another fluorescent probe which may be used in the array of the present invention. The fluorescence of IAEDANS is quite dependent upon environment, although much less so than that of IAANS and MIANS conjugates; its conjugates frequently respond to ligand binding by undergoing spectral shifts and changes in fluorescence intensity that are determined by the degree of aqueous salvation. Advantages of this reagent include high water solubility above pH 4 and a relatively long fluorescence lifetime (sometimes >20 nanoseconds, although commonly 10-15 nanoseconds). In addition, because it has a large Stokes shift and an emission that overlaps well with the absorption of fluorescein, Alexa Fluor 488, Oregon Green dyes and BODIPY FL dyes, IAEDANS is an excellent reagent for (FRET) measurements of proximity up to about 60 Å.

Most preferred fluorescent probes suitable for use as labels in the present invention are iodoacetylnitrobenzoxadiazole, coumarins and acrylodans.

ix) Detector Array Assembly

The biological sensing elements for use in the detector array of the present invention are immobilized onto or within a solid support. Typically, the sensing elements are tagged for purification and/or immobilization. Tagging must not eliminate the binding activity of the polypeptide. Exemplary tagging systems include the hexahistidine or glutathione-S-transferase tag, as are well known in the art and described herein. Once linked to such an affinity tag, the sensing element can be easily immobilized onto or within a solid matrix via the affinity tag and its ligand (for example Ni-NTA or glutathione). The sensing elements may be tagged at the N-terminus, C-terminus or even both or other locations within the polypeptide chain, so long as the tagging does not eliminate the binding activity of the sensing element.

Typically, the biological sensing elements and variants thereof are immobilized onto or in discrete regions of a solid substrate. The substrate may be porous to allow immobilization within the substrate or substantially non-porous, in which case the sensing elements are typically immobilized on the surface of the substrate. The solid substrate may be made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes, such as nitrocellulose or nylon membranes, which are widely available. The semi-permeable membranes may be mounted on a more robust solid surface such as glass. The surfaces may optionally be coated with a layer of metal, such as gold, platinum or other transition metal. A particular example of a suitable solid substrate is the commercially available surface modified glass microscope slides (Xenopore Inc.). When using a solid substrate such as a surface modified glass microscope slide, the sensing elements/variants may be applied to the slide by, for example, spotting. As described in the Examples herein, aliquots of the sensing element/variants are spotted directly onto a nickel nitrilotriacetate modified glass slide.

The solid substrate is conveniently divided up into sections. This may be achieved by techniques such as photoetching or by the application of hydrophobic inks, for example Teflon-based inks (Cel-line, USA).

Attachment of the sensing elements to the substrate may be by covalent or non-covalent means. Typically the sensing elements are attached to the substrate via a layer of molecules to which the sensing elements bind. For example, the sensing elements may be modified with biotin and the substrate coated with avidin and/or streptavidin. A convenient feature of using biotinylated sensing elements is that the efficiency of coupling to the solid substrate can be determined easily. Since the sensing elements may bind only poorly to some solid substrates, it is often necessary to provide a chemical interface between the solid substrate (such as in the case of glass) and the sensing element. Examples of suitable chemical interfaces include organofunctional silanes and long-chain thiol alkanes with terminal functional groups such as terminal carboxylic acid groups. Another example is the use of polylysine coated glass, the polylysine then being chemically modified using standard procedures to introduce an affinity ligand such as nitrilotriacetate (NTA) or biotin. Other methods for attaching molecules to the surfaces of sensor chips by the use of coupling agents are known in the art, see for example WO 98/49557.

It is desirable to confirm the efficiency of coupling using standard techniques to establish the amount of sensing element bound at each cell on the solid substrate. This is typically carried out using the detectable label by measuring the emission at a particular wavelength. This information may be used to normalize the results obtained from various positions in the array.

In a further embodiment, the present invention provides a method for producing a detector array for analyzing a ligand in a sample, comprising:

(a) selecting a broad specificity sensing element capable of binding a broad range of structurally diverse ligands;

(b) performing mutagenesis and/or chemical modification of the ligand binding site of the sensing element to produce a variant differing from the element from which it was derived in its ligand binding specificity and/or affinity;

(c) attaching a label to each sensing element and variant thereof; and

(d) immobilizing each sensing element and variant thereof discretely onto or within a solid support.

x) Assay System

The present invention also provides a method for screening a candidate ligand for a desired biological activity which comprises contacting a detector array as hereinabove defined with the candidate ligand and comparing the data array pattern thereby obtained with the reference database obtained from the data array patterns of test ligands.

The invention also provides a means to analyze a sample ligand which method comprises contacting a detector array as hereinabove defined with the sample ligand and comparing the data array pattern thereby obtained with the reference database obtained from the data array patterns of test ligands.

Thus the detector array of the present application may be used for a variety of purposes.

In a first aspect, the array may be used to analyze a complex mixture of sample ligands. Typically, a complex mixture of ligands will comprise 2 or more ligands in the mixture, wherein the ligands are either structurally related, of similar molecular weight or have similar activities/properties. It will be understood that the method may be advantageously applied to simple mixtures of ligands (which typically comprise 2 or more ligands which clearly differ in, for example, structure, or activity/properties) or to samples comprising a single species of ligand, or any combination thereof.

By analyze is meant that the data array pattern resulting from contacting the detector array of the present invention with the sample ligand, either alone or in a simple or complex mixture, is compared with a reference database formed from the data array pattern resulting from contacting the detector array of the present invention with known ligands of a similar class or activity to the sample ligand, such that the points of similarity and difference resulting from that comparison will enable an identification of the sample ligand to be made. Where the sample ligand is in mixture of two or more ligands, the method may be used to identify a particular single sample ligand, or some or all of the ligands present in the mixture. Comparisons are usually made using appropriate software.

Samples may be in gaseous, liquid or solid form (or combination thereof) such as in the form of solid samples, gaseous samples extracted from the atmosphere, liquid environmental samples (for example from a contaminated site), gaseous biological samples, such as exhaled air or liquid biological samples such as saliva, blood, serum, sweat, urine, milk, bone marrow, cerebrospinal fluid, synovial fluid, amniotic fluid or lymphatic fluid. Samples may also be volatile. Solid samples may be processed in a suitable solvent, such as water or organic solvents, to produce liquid samples. Solid samples may also by pyrolyzed to produce gaseous samples. Samples are preferably of low molecular weight.

As a first step in the detection/identification of a ligand in a sample, the detector array must first be “trained” or calibrated using a panel of known ligands. The detector array is contacted with the known ligands, normally one at a time, under specific conditions and the change in the physical characteristics of the detectable label are measured.

In a second aspect, the detector array of the present invention may be used to screen for compounds (“candidate ligands”) having a desired activity or property. This use of the detector array will have particular advantages in accelerating the screening of new compounds.

In particular, the detector array is first “trained” to recognize a specific activity or property. This may take place by contacting the array with a number of known test ligands all sharing the activity or property to be tested for, for example compounds all of which are non-steroidal anti-inflammatory agents. A reference database may then be formed from the data array patterns of those test ligands. Neural net, multivariate statistical or pattern recognition software may be used to interpret the database, such that particular characteristics demonstrated on the detector array which are common to the known test ligands and therefore associated with their common activity/property can be identified. A candidate ligand suspected of having the desired activity/property may then be screened for that activity/property by contacting the same detector array with the candidate ligand and comparing the data array pattern thereby obtained with the reference database. The use of neural net multivariate or pattern recognition software should allow a determination to be made of whether the data array pattern of the candidate ligand demonstrates the particular characteristics shown to be common to the test ligands and associated with the activity/property being tested for. This comparison should allow a determination to be made of the likelihood that the candidate compound possesses the desired activity/property.

In a third aspect, the detector array of the present invention may be used to create a surrogate proteome. The human proteome is currently estimated to consist of 100-150,000 secreted proteins and is a 3-dimensional collection of binding sites presented on a variety of protein scaffolds. Some of the binding sites within the proteome are specific, that is they cover a confined area of 3-dimensional binding space, and others are promiscuous, that is they cover a broader area of 3-dimensional binding space.

The detector array, in the form of a surrogate proteome, can be used to map molecular interactions, for example it can help identify drug selectivity, toxicity and protein partners and thereby provide further information on compound/drug-protein binding as the mediator of the transmission of biochemical and cellular signals that result in the therapeutic action of a drug. The surrogate proteome also enables a study of the cross-reactivity of a protein or drug to be made, this being with drugs the most probable cause of side effects, toxicity and compound attrition in drug development.

The advantages of such a surrogate proteome are numerous and include the following:

drugs can be classified based upon a real biological binding model which is more accurate than a computer model;

truly diverse sensing elements with unique binding properties can be recognized;

3-dimensional models of binding sites of bound orphan drugs and proteins can be made for use in target validation and pathway mapping. The structure of the binding site can be obtained using, for example, X-ray crystallography and computer models. The obtained structure can then be used to identify potential native binding proteins;

drug binding sites consistently correlated with specific side effects or toxicity can be identified; and

the binding information and patterns obtained using neural nets or other statistical or multivariate data analysis can be used to enhance or speed up the decision pathway for moving potential compounds into drug development.

xi) Measurements

A particular advantage of the array and method of the present invention is that, by contrast to other methods, it is not necessary to remove unbound constituents by washing prior to making a measurement. Thus, when the array is contacted with a ligand, measurements of the physical change in the detectable label can be made over time. This allows the dynamic changes (ΔI/Δt) in ligand binding to be measured and allows for the generation of many sets of data which will provide a characterization of the binding of the ligand to each sensing element and variant thereof.

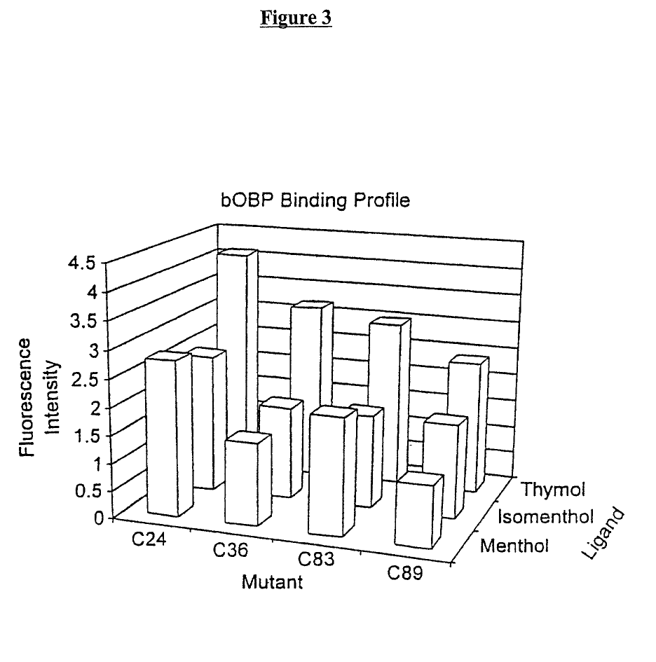

Measurement of ligand binding may be one-dimensional. In this aspect, a measurement of, for example, the change in fluorescence intensity caused by an interaction of a ligand with the binding site of a sensing element could be made at a certain point in time, that is the height of the peak on a graph could be measured at a certain point in time. Thus the maximum fluorescence intensity can be measured at a specific wavelength. In a similar manner, the change in fluorescence intensity caused by the interaction described above can be measured for each sensing element and each variant thereof. A pattern in one dimension will thus be obtained, that is a pattern demonstrating either the difference in fluorescence intensity at each sensing element and variant thereof at a single point in time or demonstrating the different maximum peaks at a certain wavelength for each sensing element and variant thereof. Reference to FIG. 3 will illustrate a one-dimensional measurement. In this Figure, the array consists of four mutants, C24, C36, C83 and C89 of bovine olfactory binding protein. Three compounds have been added to this array and the maximum fluorescence intensity measured at each mutant and for each compound. As may be seen, the maximum change in fluorescence intensity differs at each of the four mutants, thereby producing a simple data array pattern for each of the three test compounds. A visual comparison of the bar graph in FIG. 3 demonstrates that with a difference in the data array patterns for each of the three test compounds, these data may be used to distinguish one compound from the others.

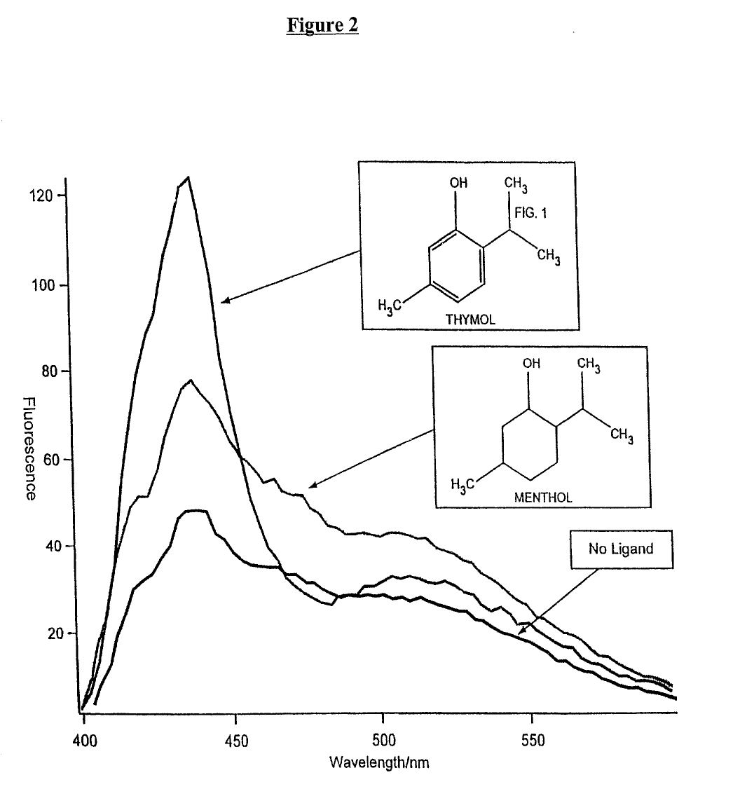

Measurement of ligand binding may be two-dimensional. Referring to FIG. 2, herein, it can be seen that measurements of the wavelength spectrum over which a peak is present can be made for each sensing element and variant thereof. The resulting data will be two-dimensional. Two-dimensional data can also be generated by measuring the time dependence (rate) of change in a fluorescence property at a single wavelength.

Alternatively, or in addition, measurement of ligand binding may be three-dimensional. Again referring to FIG. 2, it is apparent that a third dimension can be introduced by measuring both the time dependence and wavelength spectrum over which changes in a fluorescence property occur. The graph in FIG. 2 will thus stretch back into the page with the third axis being time. The resulting data will therefore be three-dimensional. In this case, the ΔI/Δt, Kd and normalization function for each ligand interacting with the sensing elements/variants can all be measured. Extension of these concepts to higher dimensions can readily be envisioned.

With so much data available, it is possible to “fingerprint” or profile a compound, that is to provide a data array pattern, formed from the binding profile of that compound on an appropriate detector array, which is specific for that compound alone. The present invention therefore includes the fingerprinting of a compound using the detector array herein described.

Measurement of ligand binding over time furthermore allows the collection of three discriminating sets of data: (a) the quantity of bound ligand; (b) the association and/or dissociation rate constants; and (c) the ligand bound to ligand free signal ratio. Each of these sets of data can be collected for each biological sensing element and each variant thereby providing a unique signature at each sensing element and variant for each ligand with which the detector array is contacted.

The complete detector array is typically read by charged coupled device (CCD) camera or confocal imaging system. The detection means are capable of interrogating each sensing element optically or electrically. Alternatively the detector array may be placed for detection in a suitable apparatus that can move in an x-y direction, such as a plate reader. In this way, the change in characteristics for each labeled sensing element can be measured automatically by computer controlled movement of the array to place each discrete element in turn in line with the detection means.

The three potential sets of data for each sensing element and variant in the array are termed a “data array pattern”. The data array pattern will generally be in the form of numerical values for each discrete element, such as in the form of a matrix, table or other data array. The results obtained may also be in visual form, such as a graphical representation (several detection methods such as spectrometry give rise to results presented as graphs), but these are preferably capable of being quantitated to provide numerical values. The use of CCD will usually result in an image made up of discrete pixels with a grey-scale or color intensity for each pixel. The pixel values are numerical data by may be displayed as grey-scale or color images.

In order to interpret the data array pattern, the pattern is stored by electronic means to give a reference database or to generate a reference library of patterns. Typically, the data are interpreted using neural net, multivariate statistical or pattern recognition software, which is advantageous since the system can be “trained” to improve its ability to discriminate between samples.

Once a reference database or reference library has been created for a panel of known test ligands, the detector array can be contacted in a similar manner with a sample or candidate ligand. Any one or all of the discriminating sets of data mentioned above are collected in a similar way for each sensing element and variant thereof. The resulting data array pattern is then fed into the neural net multivariate statistical or pattern recognition software such that a comparison between one or more test ligands and the sample or candidate ligand can be made. The software is able to obtain a best-fit. The software may also indicate the degree of statistical certainty with which the best-fit match has been made and optionally set a threshold where a sample is rejected as unknown. These techniques are well known in the art.

Further diversity in the specificity and/or affinity characteristics of the sensing elements and variants thereof can be desirable, as this may result in the obtaining of additional data further characterizing the ligand being analyzed. Such diversity may be achieved by, for example, varying the conditions under which the detector array is contacted with the sample. Thus, the temperature, pH and/or salt concentration may be altered to achieve such diversity.

The methods and arrays of the present invention may be used in a variety of different applications, such as identifying particular compounds or groups of compounds in a sample. For example, they may be used to detect pathogens, such as bacteria, fungi or viruses in environmental or biological samples. They may be used to detect molecules associated with and/or indicative of pathological states. They may also be used to detect chemical contamination in environmental samples such as air or water.

The detector array and methods of the present invention may also be used to aid classification of compounds. Thus, for example, a certain signature or data array pattern may be indicative of, for example, toxicity of a potential drug. The present array would therefore allow comparison of candidate or sample compounds with the data array pattern of compounds having known toxicity characteristics.

The detector array and methods of the present invention may also be used in the screening of known or novel compounds for a particular activity/property. Thus, for example, a data array pattern common to all compounds having a specific activity/property may be used to determine whether a known or novel compound has that activity/property.

Thus the methods and arrays of the present invention may be used in a variety of industrial, clinical and environmental applications.

The detector arrays of the present invention may be provided as kits. Such kits will typically comprise at least one detector array and optionally reagents required for standardizing reaction conditions, such as buffers. The kit may also comprise the detection means and/or analysis software, optionally comprising a database of reference sample patterns. The kit will also generally comprise instructions for using the kit.

It is an advantage of the present invention that the fluorescent group is an integral part of the individual sensing elements and variants thereof of the array. This allows for a more streamlined and/or labor saving process to be used in the analysis, alleviating the need for additional fluorescent reagents to be added either simultaneously with or after the ligand binding stage of the procedure.

xii) Computer Systems

In another aspect, the present invention provides systems, particularly a computer system, the system containing one or more of (a) 1-, 2-, 3- or higher-dimensional binding data relating to the binding of a test set of ligands to an array of the invention, (b) processed binding data defining a set of parameters associated with the binding of a test set of ligands having a desired biological property; or (c) software capable of comparing binding data of an array of a candidate ligand with the set of parameters defined as (b).

In a further aspect, the present invention provides computer readable media with (a) 1-, 2-, 3-, or higher-dimensional binding data relating to the binding of a test set of ligands to an array of the invention; (b) processed binding data defining a set of parameters associated with the binding of a test set of ligands having a desired biological property; or (c) software capable of comparing binding data to an array of a candidate ligand with the set of parameters defined as (b).

As used herein, “computer readable media” refers to any medium or media which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the binding data of a test set of ligands can be routinely accessed for analysis and comparison. Thus such analysis may include further data processing in order to generate a computer model capable of discriminating members of the test set with and without a desired activity, or comparison of a candidate ligand (i.e. a ligand under investigation for its potential to exhibit a desired biological activity) with a computer model in order to determine the likelihood (which might be expressed as a mathematical probability or merely in qualitative terms) of the candidate so exhibiting such activity.

As used herein, “a computer system” refers to the hardware means, software means and data storage means used to analyze the ligand binding data generated according to the present invention. The minimum hardware means of the computer-based systems of the present invention comprises a central processing unit (CPU), input means, output means and data storage means. Desirably a monitor is provided to visualize the raw data and its processed forms. The data storage means may be RAM or means for accessing computer readable media of the invention. Examples of such systems are microcomputer workstations available from Silicon Graphics Incorporated and Sun Microsystems running Unix based, Windows NT, MacOS or IBM OS/2 operating systems.

Thus by providing such systems, the screening of candidate ligands for a potential biological activity can be integrated and fully or partially automated in order to facilitate the process of drug discovery.

In a further aspect the invention thus provides a method for screening a candidate ligand for a desired biological activity, which method provides:

(i) providing an array according to the invention;

(ii) providing a computer system containing either or both of

(a) ligand biding data for a test set of ligands to said array which exhibit a desired biological activity; and

(b) processed binding data defining a set of parameters associated with the binding of a test set of ligands having a desired biological property;

(iii) binding a candidate ligand to the array;

(iv) measuring the binding profile of said candidate ligand to said array; and

(v) comparing the binding profile with the data of (ii) and predicting from said profile the likelihood that the ligand exhibits said desired biological activity.

In this aspect of the invention, it will be apparent to a person of skill in the art that the computer system may have been obtained separately and prior to the screening of candidate ligands. It will be possible for such a system to be generated independently of the subsequent screening process.

By utilizing this method of the invention, it will is possible to screen a large number of ligands rapidly and obtain a rapid elimination of ligands with no or low probability of exhibiting the desired activity. The ligands which are determined to be of potential interest may then be screened in more complex systems which would be impractical or cumbersome to use on a large scale with a large number of candidate ligands, or which are more difficult or expensive to configure reliably (e.g. receptor binding, cell growth or inhibition assays and the like).

The following examples are offered by way of illustration and not by way of limitation.

### EXAMPLE 1

### Generating an Array of Variants of the Maltose Binding Protein (mbp)

A nucleotide sequence encoding maltose binding protein (malE gene) is cloned into an expression vector in frame with a C-terminal hexahistidine sequence (6×His tag). The vector is the commercially available pET28b. The malE gene carries the signal peptide sequence to ensure periplasmic expression. In addition the mbp coding sequence has a mutation (for example at position 337 in the protein sequence) so that the protein has a cysteine residue.

Error prone PCR or cassette mutagenesis with mixed mutagenic primers is used to produce variants of the malE gene using the vector DNA as a template. These methods are well known in the art.

The mixture of randomly mutated DNA molecules is transformed into a suitable host strain (such as E. coli BL21 (DE3)). Transformation conditions are chosen so that each cell takes up a maximum of one molecule of DNA. The cells are then plated onto nutrient agar (containing an antibiotic for selecting only those cells which have been transformed) in a Petri dish and left to grow overnight.

Individual colonies are picked and inoculated into the wells of a microtitre plate. The cells are left to grow for a few (typically 4) hours and then half the contents of each well transferred to a new microtitre plate (the ‘master’ plate). Protein expression is induced in the first of the plates and the other (the ‘master’) is stored.

After a further period of time (typically more than 2 and less than 24 hours) the cells are centrifuged in the plates and the supernatant liquid removed. The cells are then osmotically shocked to release the contents of the periplasm. The cells are then centrifuged and the supernatant transferred to a new microtitre plate.

A fluorescent probe, such as iodoacetylnitrobenzoxadiazole, is then added to each well of the microtitre plate to label the protein via a reaction with a cysteine residue present in the protein.

A microscope slide coated with poly(lysine) is chemically modified using published procedures to introduce nitrilotriacetate groups. These are then converted to the nickel or copper complex by adding a solution of the sulfate salt of the respective metal ion.

The slide is then washed and the contents of each well are then placed onto the modified microscope slide in discrete locations and allowed to react in a humid atmosphere until binding has reached equilibrium. The slide is then washed again and is now ready for use.

### EXAMPLE 2

### Using an Array of Fluorescent Proteins

A slide carrying an array of fluorescent proteins as described in Example 1 is mounted in a flow cell such that solution comprising test compounds can be passed over its surface.

Four or more different compounds are tested with the array, either individually or in various combinations.

To measure the pattern for each compound or combination, the flow cell is placed on the stage of an epi fluorescence microscope so that it is excited by light of a wavelength that causes fluorescence of the dye attached to the sensing elements.

An image of the fluorescent light emitted from the array is collected before and after exposing the slide to a sample for analysis.

A comparison of the patterns of fluorescence before and after exposure of the slide to the samples that contain only one compound is used to initiate training of a neural net or calibration with multivariate statistical or pattern recognition algorithms implemented in software. Once the reference data have been obtained, the patterns obtained for combinations of compounds are input into the software to determine whether it can discriminate individual compounds present either singly or in a mixture. Finally, a blind study is conducted using samples containing various combinations that are not known in advance to the tester. The tester feeds the patterns obtained into the neural net software and compares the answer provided with the actual known sample composition.

### EXAMPLE 3

### Immobilization of Site Specifically Labeled Bovine Odorant Binding Protein onto Microtitre Plates

A nucleotide sequence encoding the bovine odorant binding protein (bOBP) and carrying silent mutations that optimize codon usage in the expression host (Escherichia coli,) is cloned in to the expression vector pET24a using techniques well known to those skilled in the art. A derived sequence of the native (wild type) gene is shown as SEQ.ID.No.1.

Mutations are made at positions 36 and 89 in the protein sequence using the technique of polymerase chain reaction according to the following method.

1. Prepare template. The bOBP gene is subcloned in the pBluescript vector using standard molecular biology techniques well known to those versed in the art.

This plasmid construct is then used as the template for an inverse PCR.

2. The primer set is designed such that 5′ ends of two primers are adjacent to each other. There is no overlap or gap between the two ends.

The primers contain the necessary sequence mismatch(es) to introduce one or more base mutations. The mutations may be either to introduce a cysteine residue for the purposes of labeling the bOBP with a fluorophore or to change residues in the ligand binding site.

3. The PCR reaction is performed with turbo Pfu or a similar proof reading polymerase. According to the manufacturer's instructions, the product of the PCR is a linear full-length sequence containing the sequence of pBluescript with the sequence of mutated gene inserted.

4. The restriction enzyme Dpn 1 is then added to digest the original template.

5. The mutated gene is excised from pBluescript and ligated into pET24a using standard molecular biology methods well known to those skilled in the art.

The plasmids carrying the mutant bOBP are transformed into the E.coli strain BL21 (DE3) that are then grown on a sterile solid medium (LB Agar) containing the antibiotic kanamycin such that only those cells that have the plasmid in them can grow by virtue of their resistance to this antibiotic. A single colony is picked and cultured in a 25 ml shake flask containing 6 mls of LB broth containing 55 μg/ml kanamycin, in an incubator (temperature 37° C., shaking speed 220 rpm). This primary culture is transferred to a 2500 ml flask containing 1000 mls of LB broth containing 50 μg/ml kanamycin, in an incubator (temperature 37° C., shaking speed 220 rpm). Bacterial cells are collected by centrifugation and lysed by the use of a French Press. The clarified supernatant is passed through a nickel chelate column (HisBind™, Qiagen Inc.) and the bOBP eluted with 600 mM imidazole. The purified protein is labeled with acrylodans under the conditions according to the Molecular Probes protocol: bOBP is labeled with a 3:1 mole ratio of acrylodans to protein and left at room temperature for 30 minutes.

### EXAMPLE 4

### Immobilization of Site Specifically Labeled Binding Proteins onto Microscope Slides

A nucleotide sequence encoding the bovine odorant binding protein (bOBP) and carrying silent mutations that optimize codon usage in the expression host (Escherichia coli) is cloned in to the expression vector pET24a using techniques well known to those skilled in the art. The derived sequence of the gene is shown as SEQ.ID.No.1.

Mutations are made at one of the following positions: 24, 36, 83 and 89 in the protein sequence using the technique of polymerase chain reaction according to the method given in Example 3 above. The mutations replace the natural residues with cysteine residues at these positions.

The plasmids carrying the mutant bOBP are transformed into the E.coli strain BL21 (DE3) and then grown on a sterile solid medium (LB Agar) containing the antibiotic kanamycin such that only those cells which have the plasmid in them can grow by virtue of their resistance to this antibiotic. A single colony is picked and cultured in a 25 ml shake flask containing 6 mls of LB broth containing 50 μg/ml kanamycin, in an incubator (temperature 37° C., shaking speed 220 rpm). This primary culture is transferred to a 2500 ml flask containing 1000 mls of LB broth containing 50 μg/ml kanamycin, in an incubator (temperature 37° C., shaking speed 220 rpm). Bacterial cells are collected by centrifugation and lysed by the use of a French Press. The clarified supernatant is passed through a nickel chelate column (HisBind™, Qiagen Inc.) and the bOBP eluted with 600 mM imidazole. The purified protein is labeled with acrylodans under the conditions according to the Molecular Probes protocol: bOBP is labeled with a 3:1 mole ratio of acrylodans and left for 30 minutes at room temperature.

Nickel nitrilotriacetate modified microscope slides from Xenopore Inc. are spotted with 5 μl spots of solutions of bOBP (C36), bOBP (C39), each protein labeled with acrylodans as set out above.

### EXAMPLE 5

### Screening Site Specific Cysteine Mutants of Odorant Binding Protein for Ligand Dependent Changes in the Fluorescence of an Extrinsic Fluorophore

A nucleotide sequence encoding the bovine odorant binding protein (bOBP) and carrying silent mutations that optimize codon usage in the expression host (Escherichia coli) is cloned in to the expression vector pET24a using techniques described in Example 3 above.

Mutations are made at one of the following positions: 24, 36, 83 and 89 in the protein sequence using the technique of polymerase chain reaction according to the methods described above. These mutations replace the natural residues with cysteine residues at the named positions.

The plasmids carrying the wild type or mutant bOBP are transformed into the E.coli strain BL21 (DE3) which is then grown on a sterile solid medium (LB Agar) containing the antibiotic kanamycin such that only those cells that have the plasmid in them can grow by virtue of their resistance to this antibiotic.

Single colonies are then picked from the solid medium and added to 200 ul of sterile LB medium (also containing kanamycin, LB^{K}) in the wells of a 96-well microplate. The plates are incubated at 37° C. overnight with shaking.

20 ul of this solution are then transferred to a fresh 200 ul of LB^{K} also in a microplate well. A further 6 wells in the same plate are similarly prepared.

The plate is sealed with a plastic film and placed at 37° C. with shaking for 1 hour. The sealing film is removed and 1 ul of a 1M solution of β-isopropyl thiogalactoside (IPTG) is added to each well, the plate resealed and incubated for a further 4 hours.

The sealing film is removed and 100 ul of BugBuster™ is then added to each well, the plate is resealed and incubated at room temperature for 30 minutes followed by centrifugation for 30 mins at 4000 rpm.

The contents of each well are then transferred to individual wells of a 96-well plate, which has been modified with nickel nitrilotriacetate groups (NiNTA) (Qiagen). The plate is sealed and incubated at room temperature for 1 hour. Liquid is then aspirated from the wells and each well washed 4 times with phosphate buffered saline (PBS).

After the last wash 200 ul of PBS is added to each well followed by 1 ul of a 5 mM solution of acrylodans.

The plate is then incubated for 30 minutes and washed 3 times with PBS. The wells are then filled with 200 ul of PBS and the fluorescence emission spectrum measured for each well with excitation at 360 nm.

1 ul of a solution of the ligand (9 mM thymol or menthol, dissolved in dimethylformamide) is then added into each well and the fluorescence emission spectrum again measured with identical instrument settings.

Spectra for the C24 mutant treated in this fashion are shown in FIG. 2 with and without added ligands (thymol and menthol. concentrations as above).

### EXAMPLE 6

### Discrimination between Different Ligands of bOBP with Different Variants of the Protein.

Four different cysteine mutants of bOBP are made by the method described in Example 3. Each mutant has a cysteine residue introduced at a different position (one each of positions 24, 36, 83, 89) and is subsequently labeled with acrylodans as described in Example 3. The labeled mutant proteins are then individually immobilized on separate nickel NTA microscope slides as described in Example 4. The slides are then cut into pieces and each piece placed in the wells of a 96 well microplate. Each column in the plate corresponds to a different variant. In each well one of 4 different samples is added (buffer, menthol, isomenthol, thymol) such that each row corresponds to a different sample. The well is then scanned such that the fluorescence intensity (λex 350, λem 400-600 nm) is measured at 9 different positions in each well. The average intensity in each is then calculated.

FIG. 3 shows the pattern of intensities for each ligand normalized to the signal in buffer for each protein.

### EXAMPLE 7

### Generation of an Odorant Binding Protein Biosensor Detector Array for Discriminating Non-Steroidal Anti Inflammatory Compounds From a Mixture of Terpenes and Nitroaromatic Compounds

a) Producing and characterizing discriminating odorant binding protein sensing elements:

A nucleotide sequence encoding the bovine odorant binding protein (bOBP) and carrying silent mutations that optimize codon usage in the expression host (Escherichia coli) is cloned in to the expression vector pET24a using techniques well known to those skilled in the art. The sequence of the gene is shown as SEQ.ID.No.1.

Mutations are made at each of the following positions: 22, 24, 36, 69, 89 and 119 in the protein sequence using the technique of polymerase chain reaction according to the following method.

1. Prepare template. The bOBP gene is subcloned in the pBluescript vector using standard molecular biology techniques well known to those versed in the art.

This plasmid construct is then used as the template for an inverse PCR.

2. The primer set is designed such that 5′ ends of two primers are adjacent to each other. There is no overlap or gap between the two ends.

The primers contain the necessary sequence mismatch(es) to introduce one or more base mutations. The mutations may be either to introduce a cysteine residue for the purposes of labeling the bOBP with a fluorophore or to change residues in the ligand binding site.

3. The template is amplified using turbo Pfu or a similar proof reading polymerase. According to the manufacturer's instructions, the product of the PCR is a linear full-length sequence containing the sequence of pBluescript with the sequence of mutated gene inserted.

4. The restriction enzyme Dpn 1 is then added to digest the template.

5. The mutated gene is excised from pBluescript and ligated into pET24a using standard molecular biology methods well known to those skilled in the art.

The plasmids carrying the mutant bOBP are transformed into the E.coli strain BL21 (DE3) that are then grown on a sterile solid medium (LB Agar) containing the antibiotic kanamycin such that only those cells that have the plasmid in them can grow by virtue of their resistance to this antibiotic. A single colony is picked and cultured in a 25 ml shake flask containing 6 mls of LB broth containing 55 μg/ml kanamycin, in an incubator (temperature 37° C., shaking speed 220 rpm). This primary culture is transferred to a 2500 ml flask containing 1000 mls of LB broth containing 50 μg/ml kanamycin, in an incubator (temperature 37° C., shaking speed 220 rpm). Bacterial cells are collected by centrifugation and lysed by the use of a French Press. The clarified supernatant is passed through a nickel chelate column (HisBind™, Qiagen Inc.) and the bOBP eluted with 600 mM imidazole. The purified protein is labeled with acrylodans under the conditions according to the Molecular Probes protocol: bOBP is labeled with a 3:1 mole ratio of acrylodans and left at room temperature for 30 minutes. The acrylodan-labeled mutants are then passed through a PD10 desalting column and the acrylodan-labelled bOBP eluted with buffer.

99 ul samples of the acrylodan-labeled mutants in buffer are added to individual wells of a 96-well plate and the fluorescence emission spectrum measured for each well with excitation at 360 nm.

1 ul of a solution of the ligands thymol (1 mM dissolved in dimethylformamide) and isomenthol (1 mM dissolved in dimethylformamide) is then added to the wells and the fluorescence emission spectrum is again measured with identical instrument settings. This protocol was repeated with 800 nl, 600 nl, 500 nl, 400 nl, 300 nl, 200 nl, 100 nl samples of thymol and isomenthol samples being added to wells containing 99.2 ul, 99.4 ul, 99.5 ul, 99.6 ul, 99.7 ul, 99.8 ul and 99.9 ul of the acrylodan-labeled mutants in buffer. In addition a control sample of 100 ul buffer was prepared.

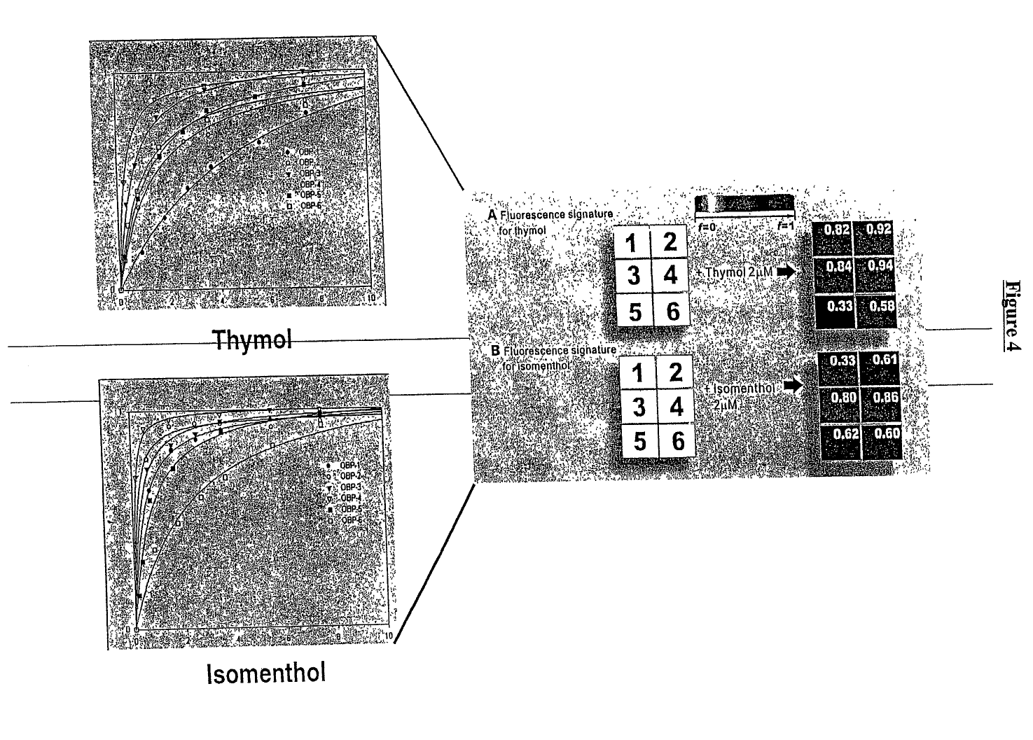

FIG. 4 illustrates the discriminating ability of the six odorant binding protein variants in solution for low molecular weight compounds of very similar structure. The graph shows that the change in the fractional saturation of the binding site of the protein variants by thymol and menthol varies with changes in ligand concentration. This indicates that the affinity-binding profile of the ligands to the sensing elements shown is both concentration and ligand dependant.

b) Production of discriminating odorant binding protein microarrays comprising six sensing element variants:

Nickel nitrilotriacetate modified microscope slides from Xenopore Inc. are spotted with 10 nl (500 um) spots of solutions of bOBP 22, bOBP 24, bOBP 36, bOBP 69, bOBP 89 and bOBP 119, each protein being labeled with acrylodans as set out above.

The arraying of the protein sensing elements is carried out on a Cartesian Microsys microarrayer using the following settings:

| | |
|---|---|
| X-Y Speed (mm/sec) | 10/50/1000 (medium) |
| Syringe Speed (ul/sec) | 8/8/800 (current) |
| Z Dispense Height (mm) | 29.50 (from top) |
| Tip Diameter (um) | 190 |
| Relative humidity (%) | 95 (24.5° C.) |
| Solenoid Dispense Valve Open Time (usec) | 200 (recommended) |

The slides containing the arrays are stored in the presence of trehalose at +4° C. ready for use in compound profiling experiments.

c) Discriminating non-steroidal anti inflammatory compounds from a mixture of terpenes and nitroaromatic compounds with odorant binding protein microarrays comprising six sensing element variants:

A slide carrying a microarray of fluorescent proteins as described above is mounted in a flow cell such that a solution comprising test compounds can be passed over its surface at a flow-rate of choice between 0.1 ml to 1 ml per minute.

Five non-steroidal anti-inflammatory compounds (NSAIDS: Naproxen, Ibuprofen, Indoprofen, Flurbiprofen and Fenoprofen) known to act as inhibitors of the cycloxidase-2 enzyme are selected to be a training compound set for the array.

To measure the affinity-binding pattern for each NSAID to the odorant binding protein array the flow cell is placed on the stage of a fluorescence imaging system. This is followed by the application of light from a laser or other light source at a wavelength that causes a fluorescence emission of the acrylodan dye attached to the protein sensing elements. A series of images over time are then collected of the fluorescent light emitted before and after exposing the slide to a compound sample for analysis.

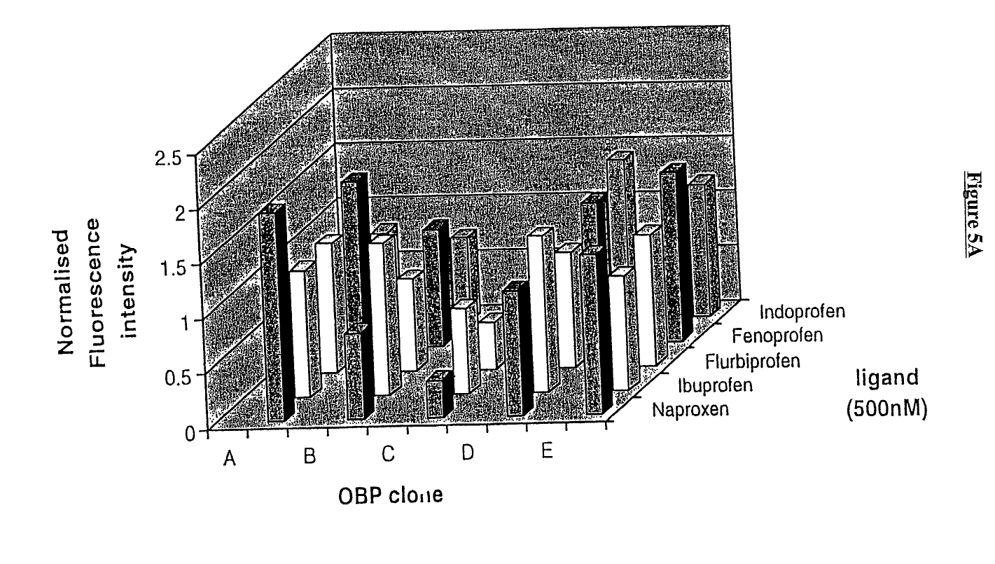

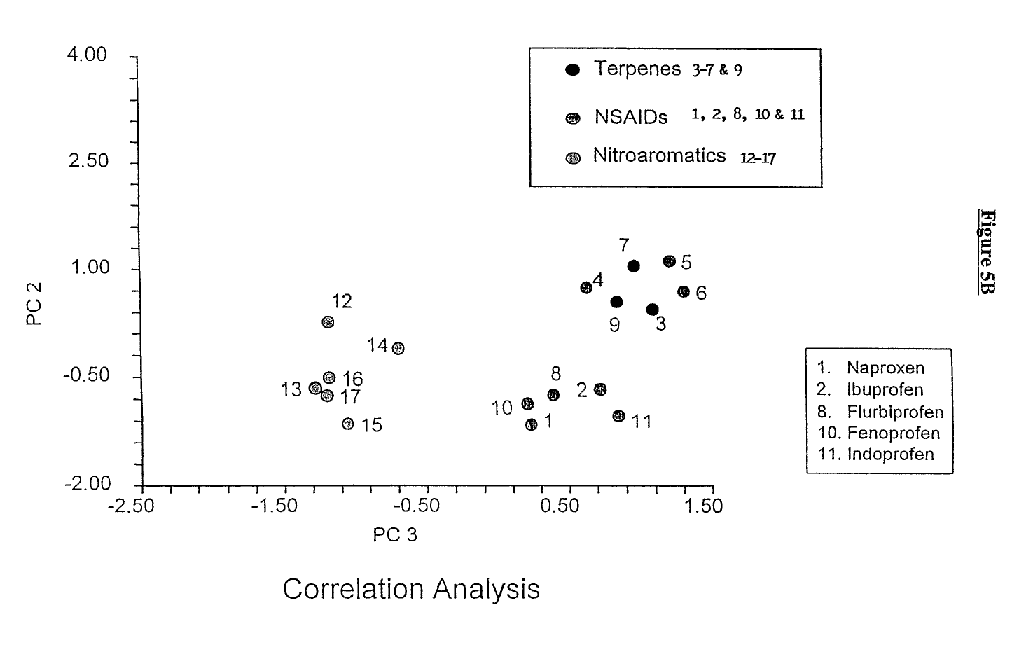

The five non-steroidal anti-inflammatory compounds are then inserted randomly into a larger set of compounds comprising 6 terpene natural products and 6 nitroaromatic compounds which are then passed over the array and their affinity-binding patterns determined. FIG. 5B shows a principle component analysis of the binding patterns of the 17 sample compounds passed over the detector array. The 17 compounds clearly cluster into three groups based upon their data array patterns enabling the discrimination of the non-steroidal anti-inflammatory group from the terpenes and nitroaromatics.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

References

Chu, F. W. and Edwards, P. R. et al. (1997) “Microarray-Based Immunoassays” Acs Symposium Series 657: 170-184.

Ekins, R. ( 1996) “Microspot(R), An Array Based Ligand Assay—Is This The Ultimate Microanalytical Technology?” Journal of Clinical Ligand Assay 19(2): 145-156.

Ekins, R. and Chu, F. W. et al. (1990) “Multispot, Multianalyte Immunoassay” Annales De Biologie Clinique 48(9): 655-666.

Ekins, R. and Chu, F. W. et al. (1990) “Multianalyte Immunoassay—The Immunological Compact-disk of the Future” Journal of Clinical Immunoassay 13(4): 169-181.

We claim:

1. A detector array comprising one or more groups of broad specificity biological sensing elements and variants thereof discretely immobilized onto or within a solid support, wherein the sensing elements and variants thereof have attached thereto a detectable label.

2. The detector array of claim 1 wherein there is at least 1 group.

3. The detector array of claim 2 wherein there are from 2 to 50 groups.

4. The detector array of claim 1 wherein each group consists of one biological sensing element and from 1 to 100 variants thereof.

5. The detector array of claim 4 wherein each group consists of one biological sensing element and from 5 to 25 variants thereof.

6. The detector array of claim 1 wherein each biological sensing element is less than 200 kDa in weight.

7. The detector array of claim 6 wherein each biological sensing element is less than 100 kDa in weight.

8. The detector array of claim 6 wherein each biological sensing element is less than 50 kDa in weight.

9. The detector array of claim 1 wherein the biological sensing element is a polypeptide or a fragment, truncation, domain or concatenation thereof comprising at least the ligand binding site of the polypeptide.

10. The detector array of claim 9 wherein the ligand binding site or sequences affected by binding of a ligand contain one or more cysteine residues.

11. The detector array of claim 9 wherein the ligand binding site or sequences affected by binding of a ligand are modified to contain one or more cysteine residues.

12. The detector array of claim 9 wherein the ligand binding site or sequences affected by binding of a ligand contain or are modified to contain one cysteine residue.

13. The detector array of claim 1 wherein a variant is derived from a biological sensing element and differs therefrom in its binding specificity and/or affinity.

14. The detector array of claim 13 wherein the biological sensing element is a polypeptide or a fragment, truncation, domain or concatenation thereof comprising at least the ligand binding site of the polypeptide.

15. The detector array of claim 14 wherein the variant contains from 1 to 5 points of difference within the amino acids sequence from the sensing element from which it was derived.

16. The detector array of claim 14 wherein the variant contains from 2 to 4 points of difference within the amino acids sequence from the sensing element from which it was derived.

17. The detector array of claim 14 wherein the difference in the binding specificity/affinity results from a difference in the amino acid composition of the ligand binding site between the sensing element and the variant thereof.

18. The detector array of claim 17 wherein the difference in amino acid composition results from chemical modification.

19. The detector array of claim 17 wherein the difference in amino acid composition results from mutagenesis.

20. The detector array of claim 1 wherein the detectable label is susceptible to change upon ligand binding.

21. The detector array of claim 1 wherein the biological sensing element is a polypeptide or a fragment, truncation, domain or concatenation thereof comprising at least the ligand binding site of the polypeptide.

22. The detector array of claim 21 wherein the detectable label is attached to a cysteine residue in the biological sensing element or variant thereof.

23. The detector array of claim 21 wherein the detectable label is attached within the ligand binding site of the biological sensing element.

24. The detector array of claim 21 wherein the detectable label is attached at different amino acid positions within the ligand binding site of the biological sensing element and within the ligand binding site of each of the variants thereof.

25. The detector array of claim 20 wherein the detectable label is a fluorophore.

26. The detector array of claim 20 wherein the detectable label is a fluorescent probe.

27. The detector array of claim 26 wherein the fluorescent probe is selected from the group consisting of acrylodans, coumarin, IANBD, IAANS, MIANS and IAEDANS.

28. The detector array of claim 1 wherein the biological sensing element is selected from the group consisting of bacterial periplasmic binding proteins, membrane proteins, odorant binding proteins from mammalian or insect olfactory organs, serum albumin, CXR protein, chaperone proteins, cytochrome P450's, P-glycoprotein, major urinary protein and DNA binding proteins.

29. The detector array of claim 28 wherein the biological sensing element is a mammalian or insect binding protein.

30. The detector array of claim 28 wherein the biological sensing element is human, bovine or porcine odorant binding protein.

31. A detector array comprising a plurality of discrete biological sensing elements immobilized onto or within a solid support wherein:
(a) each sensing element has a ligand binding site capable of binding a broad range of structurally diverse ligands;
(b) the sensing elements are provided in groups, each group comprising a biological sensing element and at least one variant thereof, said variant differing from the element from which it was derived in its ligand binding specificity and/or affinity; and
(c) each sensing element and variant thereof having a detectable label attached thereto, the physical characteristics of said label being susceptible to change upon ligand binding.

32. A method for providing a detector array system comprising:
(a) providing a detector array comprising one or more groups of broad specificity biological sensing elements and variants thereof discretely immobilized onto or within a solid support, wherein the sensing elements and variants thereof have attached thereto a detectable label;
(b) contacting the array with a panel of test ligands;
(c) measuring the characteristics of the detectable label for each sensing element and variant thereof to produce a data array pattern; and
(d) using the data array pattern to generate a reference database of said patterns.

33. The method of claim 32 wherein the measurements are of fluorescence emission spectra.

34. The method of claim 32 wherein the measurements made are time dependent.

35. The method of claim 32 wherein the detectable label is a fluorescent label.

36. The method of claim 32 further comprising the steps of:
(e) contacting the array with a sample containing one or more sample ligands;
(f) producing a data array for the ligands;
(g) comparing the data array for the sample ligands with the reference database of data array patterns obtained from said test ligands.

37. The method of claim 36 wherein the sample ligand is of low molecular weight.

38. The method of claim 36 wherein the sample is liquid or substantially gaseous.

39. The method of claim 36 wherein the sample contains volatile and/or non-volatile compounds.

40. The method of claim 36 for discriminating individual compounds present in a sample either singly or in a mixture.

41. The method of claim 36 wherein the diversity of the specificity and/or affinity characteristics of the sensing elements is altered by altering the conditions under which the array is contacted with the sample.

42. The method of any one of claims 32 to 35 wherein the test ligands comprise two or more ligands having a desired biological activity.

43. The method of claim 42 which further comprises screening the array with a candidate ligand to determine the likelihood that said candidate ligand has said biological activity.

44. A computer system, the system containing one or more of (a) 1-, 2-, 3- or higher-dimensional binding data relating to the binding of a test set of ligands to an array of the invention, (b) processed binding data defining a set of parameters associated with the binding of a test set of ligands having a desired biological property; or (c) software capable of comparing binding data of an array of a candidate ligand with the set of parameters defined as (b).

45. Computer readable media with (a) 1-, 2-, 3- or higher dimensional binding data relating to the binding of a test set of ligands to an array of the invention; (b) processed binding data defining a set of parameters associated with the binding of a test set of ligands having a desired biological property; or (c) software capable of comparing binding data to an array of a candidate ligand with the set of parameters defined as (b).

46. Use of a system according to claim 44 for screening a candidate ligand in order to determine the likelihood that the candidate has said biological property.

47. The use of a detector array as defined in claim 1 in the screening of a ligand for activity.

48. The use of a detector array as defined in claim 1 in the determination of the presence of a ligand in a sample.

49. The use of a detector array as defined in claim 1 as a surrogage proteome.

50. A method for producing a detector array for analyzing a ligand, comprising:
(a) selecting a broad specificity sensing element capable of binding a broad range of structurally diverse ligands;
(b) performing mutagenesis and/or chemical modification of the ligand binding site of the sensing element to produce a variant differing from the element from which it was derived in its ligand binding specificity and/or affinity;
(c) attaching a label to each sensing element and variant thereof; and
(d) immobilizing each sensing element and variant thereof discretely onto or within a solid support.


SEQUENCE LISTING (2 sequences)

SEQ ID NO: 1 - DNA, 524 bp, Bos taurus
ggatcctgggcgcaagagagaggaggaaagctgagcaaaatctctcagagctttcaggaccatggagaacagtgtacattgggtcgaccaacccagagaaaatccaggagaatggaccattcaggacttacttccgtgaacttgtgtttgatgatgaaaagggcacggtggacttttacttttctgtcaagcgggatggaaaatggaagaatgtacatgtcaaggctacaaagcaagacgatggtacttatgttgctgactatgagggtcaaaacgtttttaaaattgtctctctgtcgaggacgcatctggtagcacataacatcaacgtggataagcacagccagaagacagaattggccggactgtttgttaaactgaatgttgaagatgaagacttggagaaattctggaagctgacggaagacaaaggaattgacaagaaaaacgttgtgaatttcttggaaaatgaaaaccatccccaccctgaacatcatcatcatcatcatcatcatcatcattag

SEQ ID NO: 2 - DNA, 525 bp, Artificial Sequence
  Description of Artificial Sequence bOBP with 10X-His tag
ggatcctgggcgcaagaggaggaagctgagcaaaatctctcagagctttcaggaccatggagaacagtgtacattggatccaccaacccagagaaaatccaggagaatggaccattcaggacttacttccgtgaacttgtgtttgatgatgaaaagggcacagtggacttttacttttctgtcaagcgggatggaaaatggaagaatgtacatgtcaaggctacaaagcaagacgatggtacttatgttgctgactatgagggtcaaaatgtatttaaaattgtctctctgtcgaggacgcatctggtagcacataacatcaacgtggataagcacggccagaagacagaattgaccggactgtttgttaaactgaatgttgaagatgaagacttggagaaattctggaagctgacggaagacaaaggaattgacaagaaaaacgttgtgaatttcttggaaaatgaaaaccatccccaccctgaacatcatcatcatcatcatcatcatcatcattaggaattc